(19) United States
(12) Patent Application Publication
(10) Pub. No.: US 2012/0271407 A1
(43) Pub. Date: Oct. 25, 2012

(54) STENT SYSTEM HAVING INTERLOCKING SIDE EXTENSION MEMBERS

(76) Inventors: Donald K. Jones, Dripping Springs, TX (US); Vladimir Mitelberg, Austin, TX (US)

(21) Appl. No.: 13/533,909

(22) Filed: Jun. 26, 2012

Related U.S. Application Data
(63) Continuation-in-part of application No. PCT/US2011/022255, filed on Jan. 24, 2011.
(60) Provisional application No. 61/501,824, filed on Jun. 28, 2011.
(60) Provisional application No. 61/501,832, filed on Jun. 28, 2011.
(60) Provisional application No. 61/501,835, filed on Jun. 28, 2011.
(60) Provisional application No. 61/298,046, filed on Jan. 25, 2010.
(60) Provisional application No. 61/298,060, filed on Jan. 25, 2010.

(51) Int. Cl. A61F 2/82

(52) U.S. Cl. 623/1.16

(57) ABSTRACT

Devices, systems and methods are provided for performing intra-lumenal medical procedures in a desired area of the body. Stents, stent delivery devices and methods of performing medical procedures to redirect and or re-establish the intravascular flow of blood are provided for the treatment of hemorrhagic and ischemic disease states.

### CROSS-REFERENCE TO RELATED APPLICATIONS

This application claims the benefit of U.S. Provisional Application No. 61/501,824 filed Jun. 28, 2011, U.S. Provisional Application No. 61/501,832 filed Jun. 28, 2011 and U.S. Provisional Application No. 61/501,835 filed Jun. 28, 2011 all of which are hereby incorporated by reference herein in their entireties.

This application is a continuation in part of International Application No. PCT/US2011/022255 filed Jan. 24, 2011, which claims the benefit of U.S. Provisional Application No. 61/298,046 filed Jan. 25, 2010 and U.S. Provisional Application No. 61/298,060 filed Jan. 25, 2010 all of which are hereby incorporated by reference herein in their entireties.

### BACKGROUND OF THE INVENTION

The field of intralumenal therapy for the treatment of vascular disease states has for many years focused on the use of many different types of therapeutic devices. While it is currently unforeseeable that one particular device will be suitable to treat all types of vascular disease states it may however be possible to reduce the number of devices used for some disease states while at the same time improve patient outcomes at a reduced cost. To identify potential opportunities to improve the efficiency and efficacy of the devices and procedures it is important for one to understand the state of the art relative to some of the more common disease states.

For instance, one aspect of cerebrovascular disease in which the wall of a blood vessel becomes weakened. Under cerebral flow conditions the weakened vessel wall forms a bulge or aneurysm which can lead to symptomatic neurological deficits or ultimately a hemorrhagic stroke when ruptured. Once diagnosed a small number of these aneurysms are treatable from an endovascular approach using various embolization devices. These embolization devices include detachable balloons, coils, polymerizing liquids, gels, foams, stents and combinations thereof.

The most widely used embolization devices are detachable embolization coils. These coils are generally made from biologically inert platinum alloys. To treat an aneurysm, the coils are navigated to the treatment site under fluoroscopic visualization and carefully positioned within the dome of an aneurysm using sophisticated, expensive delivery systems. Typical procedures require the positioning and deployment of multiple embolization coils which are then packed to a sufficient density as to provide a mechanical impediment to flow impingement on the fragile diseased vessel wall. Some of these bare embolization coil systems have been describe in U.S. Pat. No. 5,108,407 to Geremia, et al., entitled, “Method And Apparatus For Placement Of An Embolic Coil” and U.S. Pat. No. 5,122,136 to Guglielmi, et al., entitled, “Endovascular Electrolytically Detachable Guidewire Tip For The Electroformation Of Thrombus In Arteries, Veins, Aneurysms, Vascular Malformations And Arteriovenous Fistulas.” These patents disclose devices for delivering embolic coils at predetermined positions within vessels of the human body in order to treat aneurysms, or alternatively, to occlude the blood vessel at a particular location. Many of these systems, depending on the particular location and geometry of the aneurysm, have been used to treat aneurysms with various levels of success. One drawback associated with the use of bare embolization coils relates to the inability to adequately pack or fill the aneurysm due to the geometry of the coils which can lead to long term recanalization of the aneurysm with increased risk of rupture.

Some improvements to bare embolization coils have included the incorporation of expandable foams, bioactive materials and hydrogel technology as described in the following U.S. Pat. No. 6,723,108 to Jones, et al., entitled, “Foam Matrix Embolization Device”, U.S. Pat. No. 6,423,085 to Murayama, et al., entitled, “Biodegradable Polymer Coils for Intraluminal Implants” and U.S. Pat. No. 6,238,403 to Greene, et al., entitled, “Filamentous Embolic Device with Expansible Elements.” While some of these improved embolization coils have been moderately successful in preventing or reducing the rupture and re-rupture rate of some aneurysms, the devices have their own drawbacks. For instance, in the case of bioactive coils, the materials eliciting the biological healing response are somewhat difficult to integrate with the coil structure or have mechanical properties incompatible with those of the coil making the devices difficult to accurately position within the aneurysm. In the case of some expandable foam and hydrogel technology, the expansion of the foam or hydrogel is accomplished due to an interaction of the foam or hydrogel with the surrounding blood environment. This expansion may be immediate or time delayed but is generally, at some point, out of the control of the physician. With a time delayed response the physician may find that coils which were initially placed accurately and detached become dislodged during the expansion process leading to subsequent complications.

For many aneurysms, such as wide necked or fusiform aneurysms the geometry is not suitable for coiling alone. To somewhat expand the use of embolization coils in treating some wide necked aneurysms, stent like scaffolds have been developed to provide support for coils. These types of stent like scaffolds for use in the treatment of aneurysms have been described in U.S. Pat. No. 6,605,111 to Bose et al., entitled, “Endovascular Thin Film Devices and Methods for Treating Strokes” and U.S. Pat. No. 6,673,106 to Mitelberg, et al., entitled, “Intravascular Stent Device”. While these stent like devices have broadened the types of aneurysms amenable to embolization therapy, utilization of these devices in conjunction with embolization devices is technically more complex for the physician, may involve more risk to the patient and have a substantial cost increase for the healthcare system.

To further expand the types of aneurysm suitable for interventional radiological treatment, improved stent like devices have been disclosed in U.S. Pat. No. 5,824,053 to Khosravi et al., entitled, “Helical Mesh Endoprosthesis and Method”, U.S. Pat. No. 5,951,599 to McCrory, entitled, “Occlusion System for the Endovascular Treatment of and Aneurysm” and U.S. Pat. No. 6,063,111 to Hieshima et al., entitled, “Stent Aneurysm Treatment System and Method.” When placed across the neck of an aneurysm the proposed stent like devices purport to have a sufficient density through the wall of the device to reduce flow in the aneurysm allowing the aneurysm to clot, while at the same time having a low enough density through the wall to allow small perforator vessels adjacent to the aneurysm to remain patent. Stent devices of this nature while having the potential to reduce treatment costs have not been realized commercially due to the difficulty in manufacturing, reliability in delivering the devices to the treatment site and an inability to properly position the denser portion of the stent device accurately over the neck of the aneurysm.

Another cerebrovascular disease state is ischemia resulting from reduced or blocked arterial blood flow. The arterial blockage may be due to thrombus, plaque, foreign objects or a combination thereof Generally, soft thrombus created elsewhere in the body (for example due to atrial fibrillation) that lodges in the distal cerebrovasculature may be disrupted or dissolved using mechanical devices and or thrombolytic drugs. While guidewires are typically used to disrupt the thrombus, some sophisticated thrombectomy devices have been proposed. For instance U.S. Pat. No. 4,762,130 to Fogarty et al., entitled, “Catheter with Corkscrew-Like Balloon”, U.S. Pat. No. 4,998,919 of Schepp-Pesh et al., entitled, “Thrombectomy Apparatus”, U.S. Pat. No. 5,417,703 to Brown et al., entitled “Thrombectomy Devices and Methods of Using Same”, and U.S. Pat. No. 6,663,650 to Sepetka et al., entitiled, “Systems, Methods and Devices for Removing Obstructions from a Blood Vessel” discloses devices such as catheter based corkscrew balloons, baskets or filter wires and helical coiled retrievers. Commercial and prototype versions of these devices have shown only marginal improvements over guidewires due to an inability to adequately grasp the thrombus or to gain vascular access distal to the thrombus(i.e. distal advancement of the device pushes the thrombus distally).

Plaque buildup within the lumen of the vessel, known as atherosclerotic disease, is not generally responsive to thrombolytics or mechanical disruption using guidewires. The approach to the treatment of neurovascular atherosclerotic disease has been to use modified technology developed for the treatment of cardiovascular atherosclerotic disease, such as balloons and stents, to expand the vessel at the site of the lesion to re-establish blood flow. For instance, U.S. Pat. No. 4,768,507 to Fischell et al., entitled, “Intravascular Stent and Percutaneous Insertion Catheter System for the Dilation of an Arterial Stenosis and the Prevention of Arterial Restenosis” discloses a system used for placing a coil spring stent into a vessel for the purposes of enhancing luminal dilation, preventing arterial restenosis and preventing vessel blockage resulting from intimal dissection following balloon and other methods of angioplasty. The coil spring stent is placed into spiral grooves on an insertion catheter. A back groove of the insertion catheter contains the most proximal coil of the coil spring stent which is prevented from springing radially outward by a flange. The coil spring stent is deployed when an outer cylinder is moved proximally allowing the stent to expand. Other stent systems include those disclosed in U.S. Pat. No. 4,512,338 to Balko, et al., entitled, “Process for Restoring Patency to Body Vessels”, U.S. Pat. No. 5,354,309 to Schnepp Pesch et al., entitled, “Apparatus for Widening a Body Cavity” and U.S. Pat. No. 6,833,003 to Jones et al., entitled, “Expandable Stent and Delivery System”. While the aforementioned devices may have the ability to access the cerebrovasculature, they lack sufficient structural coverage of the lesion to achieve the desired patency of the vessel without the use of a balloon device.

### SUMMARY OF THE INVENTION

In accordance with one aspect of the present invention there is provided a medical device deployment system for repairing a body lumen in a mammal. The medical device deployment system includes a stent device, a delivery system and a catheter. The stent device is positioned at the distal end of the delivery member and disposed within the lumen of the catheter. The stent device takes the form of a helically wound backbone or primary member having side extension members spaced apart along the length and extending outwardly from the backbone. The side extension members generally have two ends where one end is fixedly coupled to the backbone and the other end extending from the backbone is free, meaning it is typically uncoupled to any other structural member. As the backbone takes successive helical turns, the side extension members may be positioned adjacent to, intermesh, interlock or overlap the side extension members or backbone of subsequent or previous helical turns, generally forming a tubular structure. The adjacency, intermeshing, interlocking or overlapping side extension members create a lattice work of apertures between turns of the backbone. The size and distribution of the apertures is a function of the diameter, length and shape of the side extension members and the distance between turns of the backbone. The stent device is formed of a resilient material and has a first constrained elongate tubular configuration for delivery to a target site within a body lumen and a second unconstrained expanded tubular configuration for deployment at the target site. The delivery system includes an inner member and an outer member. The inner and outer members both have distal and proximal ends. The outer member is tubular having a lumen extending between its proximal and distal ends and is preferably torque-able. The inner member is elongate, tubular, torque-able and slidably disposed within the lumen of a tubular outer member. The distal end of the inner member extends distal to the distal end of the outer member. The stent device is mounted on the distal end of the inner member where the distal end of the stent device is secured to the distal end of the inner member by a distal electrolytically severable joint. A tip cap member is slidably position over the open distal end of the inner member covering the electrolytically severable joint. The tip cap member is secured to an elongate push wire extending through the lumen of tubular inner member and exiting its proximal end such that when the push wire is advanced distally relative to the inner member, the tip cap member slides distally relative to the distal end of the inner member thereby uncovering the electrolytically severable joint. The proximal end of the stent device is secured to the distal end of the outer member by a proximal electrolytically severable joint. Rotation of the inner member relative to the outer member in one direction causes the stent device to wind itself on to the inner member distal end while decreasing in diameter whereas rotation of the inner member in an opposite direction causes the stent device to increase in diameter expanding away from the inner member. The mounted stent device is wound to a first configuration having a reduced diameter and is positioned within the catheter lumen. The proximal ends of the inner member and outer member are maintained relative to each other so that the stent remains constrained on the inner member distal end. Additionally, provided that the inner and outer members rotate relative to each other the catheter wall will also provide a constraint to the stent device to maintain the stent in a reduced diameter. When the stent device is suitably positioned at a target site, a power supply coupled to the proximal end of the inner member provides energy through the inner member to its distal end, through the tip cap member covered distal electrolytically severable joint and stent device such that the proximal electrolytically severable joint coupled to the outer member severs, thereby releasing the stent device from the outer member. The push wire may then be advanced relative to the inner member thus sliding the tip cap member distally and uncovering the distal electrolytically severable joint. The power supply may again be activated to provide energy to the distal end of the inner member, through the uncovered distal electrolytically severable joint such that the distal electrolytically severable joint severs, thereby releasing the stent device from the inner member.

In accordance with another aspect of the present invention there is provided a stent device having a backbone and side extension members which may take various configurations comprising any of the following: side extension members on each side of the backbone which are uniformly spaced along the length of the backbone; side extension members on each side of the backbone which are not uniformly spaced along the length of the backbone; side extension members having a curved shape; side extension members having a straight shape; side extension members extending from the backbone in an angled direction; side extension members having different lengths; side extension members having apertures; side extension members having radio-opaque markers; side extension members having an enlarged tabular end; backbones having apertures; backbones having radio-opaque marker(s); backbones having a curvilinear shape.

In accordance with still another aspect of the present invention there is provided a method of reconstructing a body lumen having a defect using a stent device according to an embodiment of the present invention. The method comprises the steps of: positioning a stent device deployment system within a vessel adjacent a target site; retracting the catheter relative to the delivery system, rotating the inner member relative to the outer member thereby expanding the stent device adjacent the target site; controlling the proximity of the side extension members on one turn of the stent device relative to the side extension members on an adjacent turn of the stent device during deployment of the stent adjacent the target site; releasing the stent device from the outer member distal end electrolytically while the tip cap member covers the electrolytically severable joint at the distal end of the inner member; removing the tip cap member from covering the electrolytically severable joint at the distal end of the inner member; and, releasing the stent device from the inner member distal end electrolytically.

In accordance with still another aspect of the present invention there is provided a method of reconstructing a body lumen having a defect, such as an aneurysm, using a stent device according to an embodiment of the present invention in conjunction with embolization devices, such as embolic coils, mesh and mesh-like baskets or frameworks. The method comprises the steps of: providing a stent device having a configuration adapted to allow the delivery of an embolization device through the side wall of the stent when said stent is in a deployed configuration; positioning a stent device deployment system having a delivery system and a catheter within a vessel adjacent a target site; retracting the catheter relative to the delivery system, deploying the stent device adjacent the target site by rotating a member of said delivery system; controlling the proximity of the side extension members on adjacent turns of the stent device during deployment of the stent adjacent the target site; releasing the stent device from the outer member distal end electrolytically while the tip cap member covers the electrolytically severable joint at the distal end of the inner member; removing the tip cap member from covering the electrolytically severable joint at the distal end of the inner member; releasing the stent device from the inner member distal end electrolytically; positioning an embolization delivery system through the wall of the deployed stent; delivering an embolization device to the aneurysm wherein said embolization device is supported by the stent device; releasing said embolization device.

In accordance with still another aspect of the present invention there is provided a method of reconstructing a body lumen having a defect, such as an aneurysm, using a stent device according to an embodiment of the present invention in conjunction with embolization devices, such as embolic coils, mesh and mesh-like baskets or frameworks. The method comprises the steps of: delivering an embolization device within an aneurysm; positioning a stent device deployment system having a delivery system and a catheter within a vessel adjacent a target site; retracting the catheter relative to the delivery system, deploying the stent device adjacent the target site by rotating a member of said delivery system; controlling the proximity of the side extension members on adjacent turns of the stent device during deployment of the stent adjacent the target site; releasing the stent device from the outer member distal end electrolytically while the tip cap member covers the electrolytically severable joint at the distal end of the inner member; removing the tip cap member from covering the electrolytically severable joint at the distal end of the inner member; releasing the stent device from the inner member distal end electrolytically; and, releasing said embolization device wherein said embolization device is supported by the stent device.

In accordance with yet another aspect of the present invention there is provided a reconstruction device having first and second configurations for delivery and deployment, respectively, where the reconstruction device is operable between the first and second configurations. The reconstruction device further including a primary member having a helical shape and a plurality of extension members with each extension member having first and second ends where one of the first and second ends is fixedly coupled to the primary member and the other end is uncoupled to any other member of said reconstruction device.

In accordance with yet another aspect of the present invention there is provided a reconstruction device having first and second configurations for delivery and deployment, respectively, where the reconstruction device is operable between the first and second configurations. The reconstruction device further including a primary member having a helical shape and a plurality of extension members with each extension member having first and second ends and a body portion between said ends, where one of the first and second ends is fixedly coupled to the primary member and the body portion or other end is uncoupled to any other member of said reconstruction device that interconnects with said backbone.

In accordance with yet another aspect of the present invention there is provided a reconstruction device having first and second configurations for delivery and deployment, respectively, where the reconstruction device is operable between the first and second configurations. The reconstruction device further including a primary member having a helical shape and a plurality of extension members with each extension member having first and second end portions and a body portion between said end portions, where one of the first and second end portions is fixedly coupled to the primary member and the body portion or other end is uncoupled to any other member of said reconstruction device that interconnects with said backbone.

In accordance with still yet another aspect of the present invention there is provided a reconstruction device wherein the primary helical member is formed of a resilient non-absorbable non-erodible material and a plurality of the extension members are formed of an absorbable or bio-erodible material.

In accordance with still yet another aspect of the present invention there is provided a reconstruction device wherein the primary helical member is formed of a resilient material and includes an absorbable and or erodible material and a plurality of the extension members are formed of a resilient material and includes an absorbable and or erodible material.

In accordance with yet still another aspect of the present invention there is provided a reconstruction device comprising a biocompatible material. Suitable resilient materials include metal alloys such as Nitinol(NiTi), titanium, chromium alloy, stainless steel. Additional materials include polymers such as polyolefins, polyimides, polyamides, fluoropolymers, polyetheretherketone(PEEK), cross-linked PVA hydrogel, polytetrafluoroethylene (PTFE), expanded polytetrafluoroethylene (ePTFE), porous high density polyethylene (HDPE), polyurethane, and polyethylene terephthalate, or biodegradable materials such as polylactide polymers and polyglycolide polymers or copolymers thereof and shape memory polymers. The medical device may comprise numerous materials depending on the intended function of the device. These materials may be formed into desired shapes or attached to the device by a variety of methods which are appropriate to the materials being utilized such as laser cutting, injection molding, spray coating and casting.

In accordance with another aspect of the present invention there is provided a reconstruction device having a coating formed of a biocompatible, bioerodible and biodegradable synthetic material. The coating may further comprise one or more pharmaceutical substances or drug compositions for delivering to the tissues adjacent to the site of implantation, and one or more ligands, such as peptides which bind to cell surface receptors, small and/or large molecules, and/or antibodies or combinations thereof for capturing and immobilizing, in particular progenitor endothelial cells on the blood contacting surface of the medical device.

In accordance with yet another aspect of the present invention there is provided a delivery system having elongate inner and outer members which includes tip markers at the distal ends of the inner and outer member and a stent positioning marker located on the inner member proximal to the tip marker.

In accordance with still another aspect of the present invention there is provided a method of reconstructing a body lumen having a defect, such as an atherosclerotic lesion, using a stent device according to an embodiment of the present invention. The method comprises the steps of: providing a stent device having a configuration adapted to treat the lesion in a deployed configuration; positioning a stent device deployment system having a delivery system and a catheter within a vessel adjacent a target site; retracting the catheter relative to the delivery system, deploying the stent device adjacent the target site by rotating a member of said delivery system; controlling the proximity of the side extension members on adjacent turns of the stent device during deployment of the stent adjacent the target site; releasing the stent device from the outer member distal end electrolytically while the tip cap member covers the electrolytically severable joint at the distal end of the inner member; removing the tip cap member from covering the electrolytically severable joint at the distal end of the inner member; and, releasing the stent device from the inner member distal end electrolytically.

### BRIEF DESCRIPTION OF THE DRAWINGS

FIG. 1 is a partial cross-sectional of a stent deployment system according to an embodiment of the present invention.

FIG. 2A through 2D are enlarged partial cross-sectional views of the proximal end and distal end of the stent deployment system according to an embodiment of the present invention.

FIG. 3 is a side view of a deployed stent device according to an embodiment of the present invention.

FIGS. 4A through 4L are partial flat pattern views of stent devices according to embodiments of the present invention.

FIGS. 5A through 5F are partial cross-sectional views illustrating a method of delivering and deploying a stent device within a vessel at a target site adjacent an aneurysm according to an embodiment of the present invention.

FIGS. 6A through 6F are partial cross-sectional views illustrating a method of delivering and deploying a stent device within a vessel at a target site adjacent an atherosclerotic lesion according to an embodiment of the present invention.

### DETAILED DESCRIPTION OF THE INVENTION

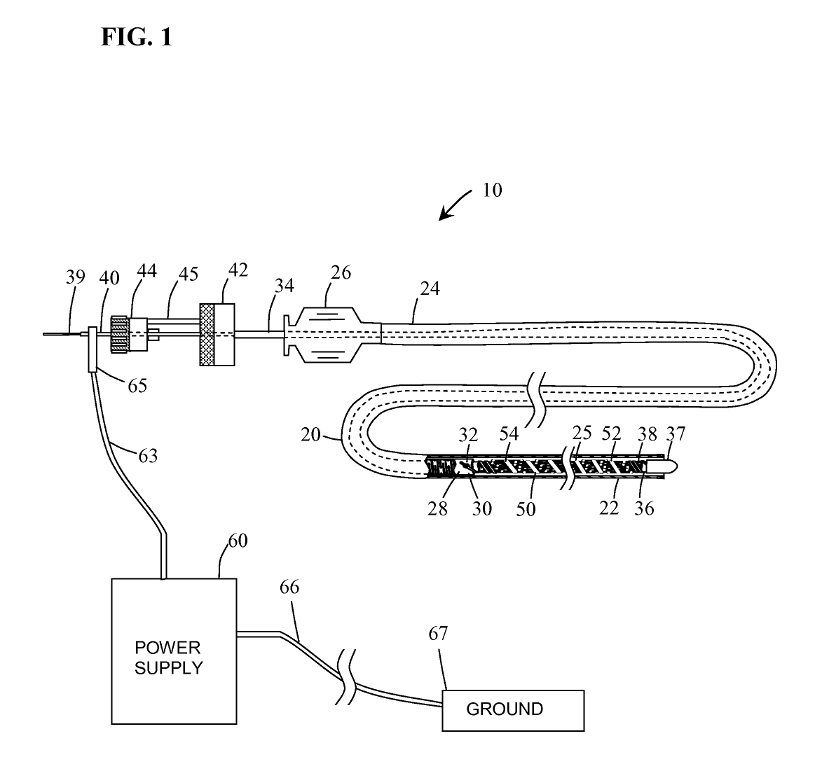

Methods and systems for performing vascular reconstruction and revascularization in a desired area of the body are herein described. FIG. 1 illustrates a medical device deployment system 10 according to an embodiment of the present invention. System 10 includes a catheter 20 having distal and proximal ends 22 and 24 respectively and a lumen 25 extending there through. Coupled to proximal end 24 is a catheter hub 26 that has a standard Luer fitting. Positioned within lumen 25 is an elongate delivery system 28 comprising an elongate tubular outer member 30 having distal and proximal ends 32 and 34 and an elongate tubular inner member 36. A tip cap member 37 is disposed on inner member distal end 38. An elongate cap push wire 39 is coupled to cap member 37 and extends proximally through the lumen of inner member 36 and proximal to inner member proximal end 40 Inner member 36 is slidably and rotatably positioned within the lumen of tubular outer member 30. The distal end 38 of inner member 36 is positioned distal to distal end 32 of outer member 30. The proximal end 40 of inner member 36 extends proximal to proximal end 34 of outer member 30. Coupled to proximal end 34 of outer member 30 is outer knob 42. Coupled to proximal end 40 of inner member 36 is inner knob 44. A retainer member 45 is removably coupled to inner knob 44 and outer knob 42 restricts rotational and axial movement of inner member 36 relative to outer member 30 until removed. The inner and outer knobs 44 and 42 together with the retainer member 45 and proximal ends 40 and 34 of the inner and outer member 36 and 30 generally constitute a rudimentary handle assembly for delivery system 28. As can be appreciated a more stylistic handle with additional features is contemplated. Stent device 50 is mounted on distal end 38 of inner member 36 and positioned within lumen 25 at catheter distal end 22. Stent device 50 has a distal portion 52 and a proximal portion 54. The deployment system 10 also includes a power supply having 60 having a lead 63 and electrode connector 65 that couples to proximal end 40 of inner member 36. A ground lead 66 and electrode pad 67 are also coupled to power supply 60. For use, electrode pad 67 is generally coupled to the patient.

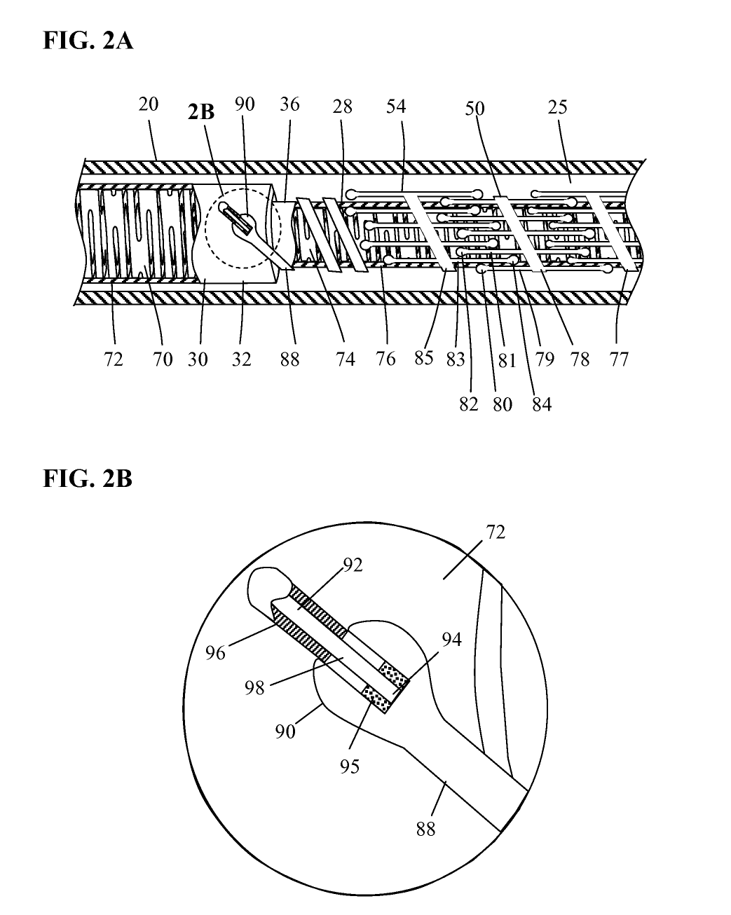

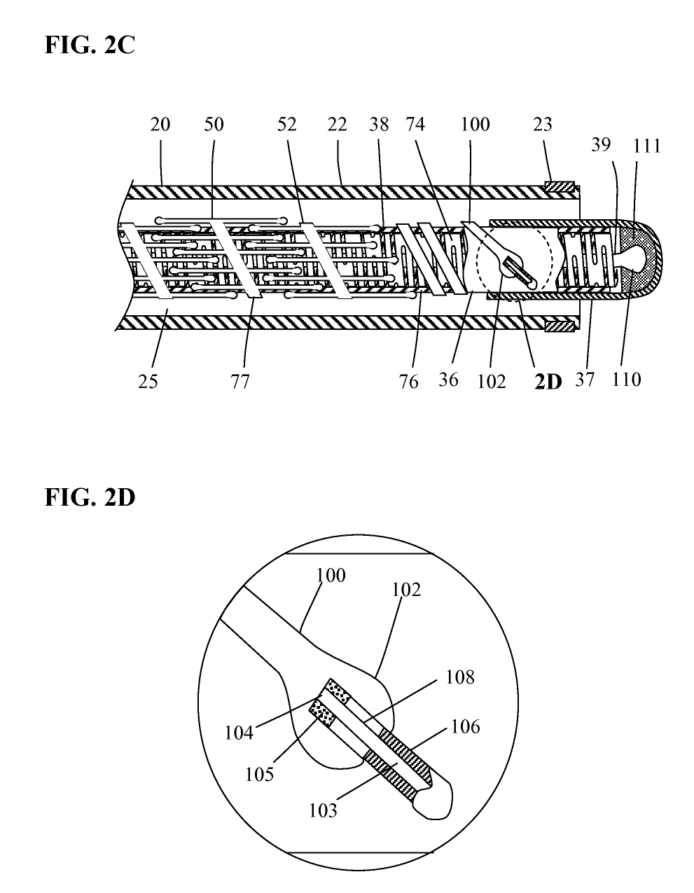

FIG. 2A illustrates an enlarged partial cross-sectional view of catheter distal end 22. Slidably positioned within lumen 25 of catheter 20 are outer member 30 and inner member 36 of delivery system 28. Outer member 30 is shown partially sectioned to reveal an internal support member 70 preferably formed as a laser cut metallic hypotube to provide flexibility and torque-ability. Alternatively, the internal support member may take the form of a wound coil assembly using wire having a round, flat or other cross-sectional shape. Support member 70 preferably has an electrically insulative cover member 72 extending over a substantial portion of the surface along its length. Cover member 72 may take the form of a thin conformal coating or shrink tubing Inner member 36 also includes a support member 74 that is preferably formed as a laser cut metallic hypotube to provide flexibility and torque-ability. Support member 74 may alternatively take the form of a torqueable wire or cable assembly. Support member 74 includes an insulative cover member 76 that extends over a substantial portion of the surface along its length. Cover member 76 may take the form of a thin conformal coating or shrink tubing. Suitable coating and shrink tubing materials include insulative polymers such as parylene, polyimides, polyamides, fluoropolymers, polyolefins, polyesters, polysiloxanes including co-polymers and composites thereof

The proximal portion 54 of stent device 50 is shown in a first configuration having a reduced diameter substantially positioned over the insulative cover member 76. Primary member 77 is shown wound around inner member 36 producing a number of turns or winds such as wind 78. Representative side extension members 79 and 81, having enlarged tabular ends 80 and 82, extend from wind 78 of primary member 77 in a direction generally parallel to the longitudinal axis of delivery system 28. Representative side extension member 83 having an enlarged tabular end 84 extends from adjacent wind 85 in a direction generally parallel to the longitudinal axis of delivery system 28 and is positioned between side extension members 79 and 81 in an intermeshing configuration. The orientation of side extension members in a longitudinal direction parallel to the longitudinal axis of the delivery system allows stent 50 be reduced to a very small diameter for positioning in a small diameter catheter having the ability to access small diameter vessels. Proximal end 88 of stent device 50 is shown having no side extension members and includes a proximal tab 90. Tab 90 is connected to distal end 32 of outer member 30 by an electrolytically severable joint member 92 at joint end 94 as shown in magnified view FIG. 2B. Joint member 92 extends through insulative cover member 72 and is in electrical communication with support member 70. Joint member 92 may be joined to support member 70 by soldering or welding (not shown). Joint end 94 is electrically coupled to tab 90 through the use of solder 95. Other means of joining joint end 94 to tab 90 may also be suitable such as forms of brazing or welding including laser welding and the use of electro-conductive adhesives. Joint member 92 includes an insulative cover 96 over the end coupled to support member 70. Joint member 92 has an exposed portion 98 that does not have an insulative covering.

FIG. 2C illustrates another enlarged partial cross-sectional view of catheter distal end 22. The distal portion 52 of stent device 50 is shown in a first configuration having a reduced diameter substantially positioned over the insulative cover member 76. Distal end 100 of stent device 50 is shown having no side extension members and includes a distal tab 102. Tab 102 is connected to distal end 38 of inner member 36 by an electrolytically severable joint member 103 at joint end 104 as shown in magnified view FIG. 2D. Joint member 103 extends through insulative cover member 76 and is in electrical communication with support member 74. Joint member 103 may be joined to support member 74 by soldering or welding (not shown). Joint end 104 is electrically coupled to tab 102 through the use of solder 105. Other means of joining joint end 104 to tab 102 may also be suitable such as forms of brazing or welding including laser welding and the use of electro-conductive adhesives. Joint member 103 includes an insulative cover 106 over the end coupled to support member 74. Joint member 103 has an exposed portion 108 that does not have an insulative covering. Once secured to joint members 92 and 103, stent device 50 is coated using an insulative coating such as parylene. This coating ensures that the exposed portions 98 and 108 of joint members 92 and 103 are the most susceptible portions for electrolytic dissolution when stent device 50 released at a target site by supplying power to delivery system 28. FIG. 2C also shows a cross section of tip cap member 37 slidably positioned on the distal end of inner member 36. Tip cap member 37 is fixedly coupled to push wire 39 by potting push wire bead 110 with adhesive 111. Tip cap member 37 has a first position that generally covers joint member 103 and a second position, distal to the first position, where joint member 103 is uncovered. Tip cap member 37 is moveable between the first and second positions by advancing or retracting push wire 39 relative to inner member 36. When tip cap member 37 is in the first position covering joint member 103, joint member 103 is generally not susceptible to electrolytic dissolution. When tip cap member 37 is in the second position where joint member 103 is not covered, joint member 103 is generally susceptible to electrolytic dissolution.

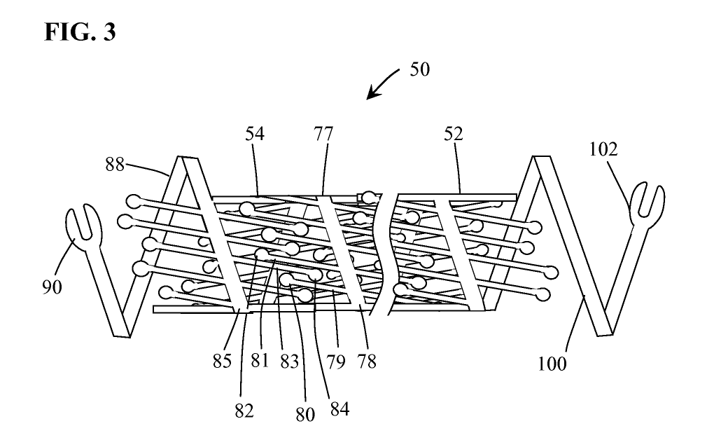

FIG. 3 illustrates detail of stent device 50 in a second configuration having an expanded diameter. The backbone or primary member 77 is shown in having helical shape along with a plurality of side extension members and turns or winds represented by side extension members 79, 81 and 83 having enlarged tabular ends 80, 82 and 84 and winds 78 and 85. As depicted, the side extension members of the stent device generally have one end secured to the backbone and the other end uncoupled which is unlike previous stents described in the art. This configuration allows the side extension members of the present invention to act as individual cantilevers providing an improved ability to conform to discrete contours within the vasculature. Alternatively, both ends of the side extension members may be coupled to the backbone or primary member, forming a looped structure for example, as long as the side extension member is discrete and not fixedly coupled to any other structural member. Prior art helical stents formed of a ladder or mesh structure in which side extension members do not have a free end or are not discrete, such as those described in U.S. Pat. No. 6,660,032 to Klumb et al, entitled, “Expandable Coil Endoluminal Prosthesis” or U.S. Pat. No. 5,824,053 to Koshravi et al, entitled “Helical Mesh Endoprosthesis and Method of Use”, do not have the same ability to conform to discrete contours of a lesion within the vasculature and instead form a wide area “tented” surface. In the expanded diameter second configuration of stent device 50 the side extension members that extended generally parallel to the longitudinal axis of the delivery system in the reduced diameter first configuration of stent device 50 are oriented at an angle to the longitudinal axis of the delivery system. Stent device 50 is shown with side extension members 79 and 81 of wind 78 intermeshing with side extension member 83 of the adjacent wind 85. The intermeshing of these side extension members creates interstices or apertures between the side extension members. The size, shape and distribution of the interstices is dependant upon the size, shape and distribution of the side extension members of a wind and an adjacent wind side extension members and the degree of intermeshing defined in part by the pitch of the backbone or primary member 77.

Generally, stents may be susceptible to migration when placed in the vasculature due to a number of factors. Some of these factors include the dimensions of the stent relative to the vessel diameter, vessel structure, degree of pulsatility, blood flow rates, stent radial resistive force, stent chronic outward force and stent geometry to name a few. In examining some of these factors in relation to a particular stent designs, such as a helical stent design, peristaltic or pulsatile motion in the vasculature may cause the stent to elongate by increasing the distance between adjacent winds thus causing an associated reduction in diameter thereby allowing the stent to migrate. Stent device 50 is less susceptible to this potential cause of migration due to the structural interlocking design of the side extension members. When subjected to peristaltic or pulsatile forces the distance between adjacent winds of stent device 50 begin to increase along with previously discussed intermeshed representative side extension members 79, 81 and 83. The enlarged tabular members 80, 82 engage and interlock with enlarged tabular member 84 to restrict the separation between the intermeshed side extension members and subsequently the adjacent winds.

The stent device 50 may have a constant diameter in the range of 1 to 50 mm, and preferably between 2 and 15 mm or as shown in FIG. 3, have ends 100 and 88 that are larger in diameter relative to other stent device portions. The diameters of the side extension members have a range of between 0.0001 and 0.025 inches with a preferred range of between 0.002 to 0.010 inches. The spacing between side extension members range between 0.001 and 0.250 inches with a preferred range between 0.002 and 0.060 inches. Stent device 50 may have regions such as ends 100 and 88 that do no have any side extension members. The wound pitch of primary member 77 is shown to be fairly constant however the pitch may be varied along a portion of a stent device dependant upon the functional requirements of the stent. For instance, a primary member having a small pitch may cause the intermeshing side extension members to have a small interstice between the tip of the side extension member and the primary member of an adjacent wind reducing the stent device porosity. Additionally, a primary member having a small pitch may cause the side extension members of one wind to overlap with the primary member of an adjacent wind.

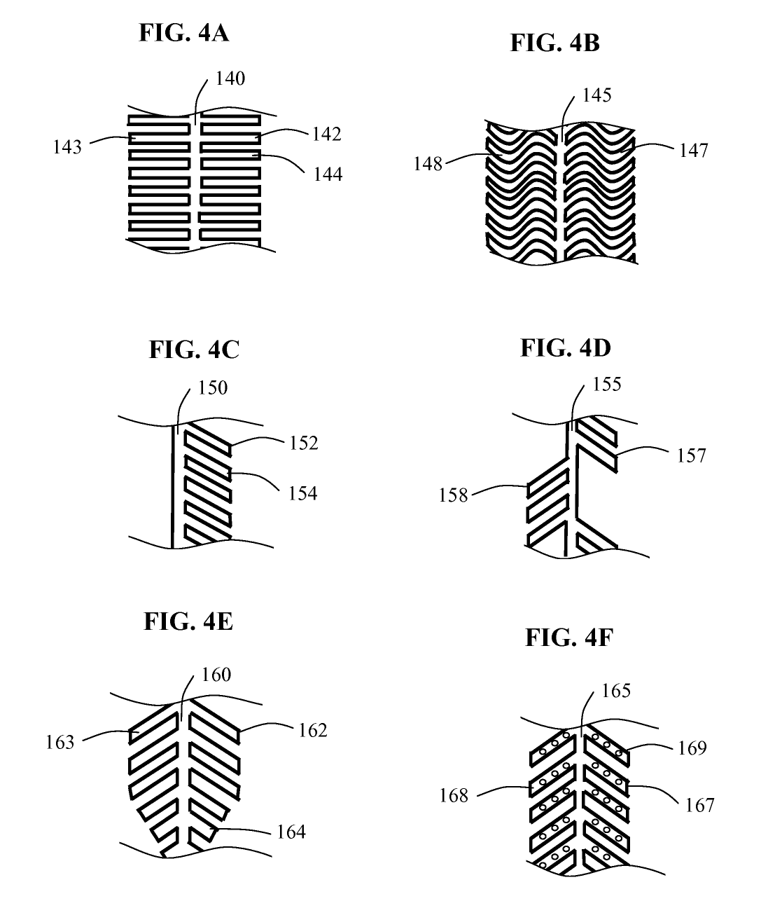

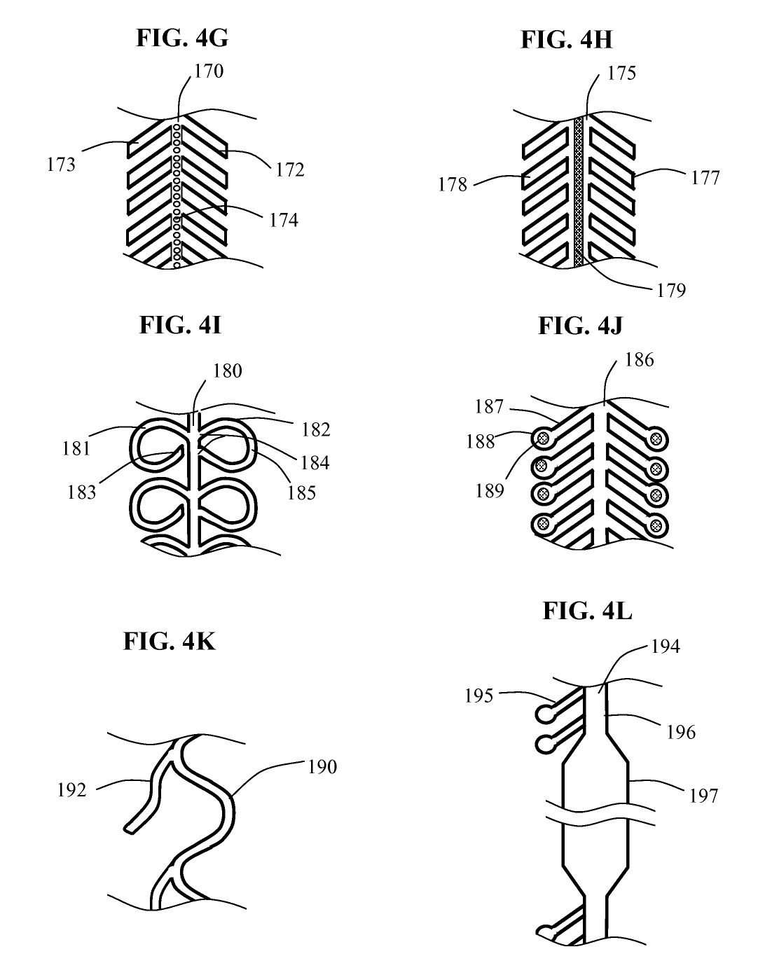

In addition to the pitch of the stent backbone having an influence on the overall porosity and porosity distribution of the stent device there exists numerous variations in the size shape and distribution of side extension members that may also influence porosity. FIGS. 4A through 4E illustrate partial flat patterns of some variations of side extension members relative to a backbone that may affect different aspects of stent performance including porosity and porosity distribution when formed in a helical shape. In one pattern variation shown in FIG. 4A, a stent device has a primary backbone 140 with side extension members 142 and 143, having generally similar diameters and lengths, extending from opposite sides of backbone 140. Side extension member 144, positioned adjacent extension member 142 has a similar length to extension 142 however may have a smaller diameter. The alternating pattern of side extension members having different diameters may be extended along backbone 140. FIG. 4B depicts another pattern variation in which a stent device has a primary backbone 145 and side extension members 147 and 148, with generally similar diameters and lengths extending from opposite sides of backbone 145 in a curvilinear shape. FIG. 4C illustrates another pattern variation in which a stent device has a primary backbone 150 and side extension members 152 and 154 which are positioned on only one side of the backbone 150. FIG. 4D shows still another pattern variation in which a stent device has a primary backbone 155 and groups of side extension members 157 and 158 are positioned in an alternating configuration on opposite sides of the backbone 155. FIG. 4E depicts still another pattern variation in which a stent device has a primary backbone 160 and side extension members 162 and 163 with generally similar diameters and lengths extending from opposite sides of backbone 160. Additionally, the side extension members may progressively have shorter lengths, such as side extension member 164, to provide a tapered configuration. FIG. 4F illustrates yet still another pattern in which a stent device has a primary backbone 165 and side extension members, 167 and 168 with generally similar diameters and lengths extending from opposite sides of backbone 165. Additionally the side extension members contain apertures 169. FIGS. 4G and 4H illustrate partial flat patterns of some variations of a backbone relative to side extension members that may affect different aspects of stent performance including porosity and porosity distribution as well as radiographic visibility when formed in a helical shape. FIG. 4G depicts a pattern of a stent device that has a primary backbone 170 and side extension members 172 and 173, with generally similar diameters and lengths extending from opposite sides of backbone 170. Along the length of backbone 170 there is a plurality of apertures 174. FIG. 4H depicts a pattern of a stent device that has a primary backbone 175 and side extension members 177 and 178, with generally similar diameters and lengths extending from opposite sides of backbone 175. Along the length of backbone 175 there is a radio-opaque member 179. The radio-opaque member 179 provides fluoroscopic visualization of the stent during the deployment procedure. For a stent device having an expanded diameter and a pre-set initial overlap of side extension members upon each helical turn of the backbone, the radio-opaque member 179 provides a visual indication of the stent pitch. As the spacing between adjacent turns of radio-opaque member 179 decreases, the amount of side extension member overlap with adjacent turns increases. FIG. 4I depicts yet another pattern of a stent device that has a primary backbone 180 and a plurality of side extension members represented by side extension members 181 and 182. Side extension members 181 and 182 are positioned on opposite sides of backbone 180 in a generally mirrored fashion for this configuration. Side extension members 181 generally take the form of an open ended loop, where a first end of the extension member loop is connected to the backbone and the second end 183 is adjacent the backbone but are not connected. Side extension members 182 generally take the form of a closed loop, where two ends 184 of the extension member loop are connected to backbone 180. As can be appreciated, side extension members 182 form a discrete side unit where it is unconnected to other side extension members except through the backbone 180. From a broader perspective two ends 184 may be considered as a first end region coupled to the backbone and a second end region 185, as shown, is free or uncoupled to any other structural member. While these loops are shown generally “circular”, the size and shape of the loop may take the form of other geometric shapes and patterns to be commensurate with the desired properties of the formed stent. For instance the loops may be rectangular, triangular or form a flattened spiral. FIG. 4J illustrates another pattern of a stent device according to an embodiment of the present invention that has a primary backbone 186 and a representative side extension member 187. While a first end of side extension member 187 is integrally coupled to backbone 186, the second end of the side extension member is uncoupled to the backbone and takes the form of an enlarged tabular end 188. This tabular end 188 is preferably rounded as to be atraumatic to the vessel wall and may include a marker element 189. Preferably marker element 189 is radio-opaque for use in fluoroscopy using known materials such as gold, platinum, tantalum, tungsten, etc., however marker materials suitable for direct visual or magnetic resonance imaging are also contemplated. Marker element 189 may be formed using coining techniques in which a round marker is press fit into a slightly smaller opening positioned on tabular end 188. Alternatively, marker 189 may be printed, coated, electro-deposited, riveted, glued, recessed or raised relative to tabular end 188. More broadly, an entire stent device or portion thereof may be coated with a radio-opaque material to provide visibility under fluoroscopy. While the marker shown in FIG. 4J is positioned at tabular end 188, the marker may be positioned at any location on the side extension member. For instance the side extension member may take the form of a threaded member and a marker take the form of a coil that is wound over the side extension member. FIG. 4K depicts still yet another flat pattern of a stent device in which the backbone 190 takes a curvilinear shape. For representative simplicity, backbone 190 is shown as being somewhat sinusoidal. Side extension member 192, also shown to be curvilinear, extends from a peak on backbone 190. As can be appreciated, side extension members such as side extension member 192 may extend from different locations on backbone 190. FIG. 4L illustrates a stent pattern where backbone 194 has side extension members represented by side extension member 195. Along its length, backbone 194 has a first width 196 and a second width 197. To impart some stretch resistance for the finished stent width 197 is shown to be greater than width 196. The amount of stretch resistance imparted in the finished stent is related to the relative difference between the two widths. The larger width may range from 1.01 to 100 times the width of the smaller width with a preferable range of 1.5 to 20 times. While FIG. 4L shows two such differing widths of the backbone, a stent may have multiple regions of differing width to make the stent suitable for a particular anatomy and clinical application. As with any of the aforementioned stent device pattern variations, these patterns may extend along the entire length of the backbone or only a portion thereof and in some instances features of various patterns may be provided in a combined fashion to form stent devices having unique performance characteristics. Preferably stent devices of the present invention comprise a biocompatible resilient material. Suitable resilient materials include metal alloys such as nitinol, titanium, stainless steel. Additional suitable materials include polymers such as polyimides, polyamides, fluoropolymers, polyetheretherketone(PEEK) and shape memory polymers. As can be appreciated, embodiments of stent devices of the present invention may be formed in part or entirely of bioabsorbable and or bioerodible materials such as polycaprolactone (PCL), polyglycolic acid (PGA), polydioxanone (PDO) and combinations thereof to allow the stent to temporarily serve structural clinical applications, deliver pharmacological compounds and then dissolve over time. These materials may be formed into desired shapes by a variety of methods which are appropriate to the materials being utilized such as laser cutting, thermal heat treating, vacuum deposition, electro-deposition, vapor deposition, chemical etching, photo-chemical etching, electro etching, stamping, injection molding, casting or any combination thereof. Preferably the stent backbone and the side extension members are integrally formed. The distance a side extension member extends from the backbone is dependant upon a specific stent design but a typical range includes between 0.5 to 100 times the width of the backbone and a preferred range being about 0.75 to 25 times the backbone width. The backbone widths have a general range of about 0.0005 in to 0.250 in with a preferred range of about 0.001 in to 0.100 in. While various configurations of side extension members, backbones and a discussion of pitch have been provided, the features of a particular stent design features are heavily dependant upon the clinical application and location of the stent. For instance, stents placed in vessels known to exhibit substantial pulsatility may require that the stent be designed to have end regions which are larger in diameter than the middle portion of the stent to better anchor the stent at the target location. Additionally, the width of the backbone may vary to provide regions of the stent which are less susceptible to elongation, thereby creating a stent that has localized stretch resistant properties which aids in reducing stent migration. Stents sufficient for treating an aneurysm without the aid of other embolization devices positioned within the aneurysm may require that the porosity of the deployed stent in the region of the aneurysm neck be less than about 30 percent. Additionally, stents for treating aneurysm in certain locations may require that the porosity across the neck be less than 30 percent however the porosity adjacent either side of the aneurysm neck be greater than 40 percent and have dimensions as not to occlude small perforator vessels adjacent the aneurysm neck. Stents used to treat fusiform aneurysms may be considerably longer than stents for berry aneurysms. Stents for use in treating a stenotic lesion may require more or less than 50 percent porosity however side member geometry should be designed to keep fragmented plaque trapped between the exterior wall of the stent and interior wall of the vessel.

As previously discussed, a specific stent device design is heavily dependant upon the clinical application for the device and may include materials or coatings to improve the biocompatibility of the device such as coatings that include ligands adapted to capture endothelial progenitor cells within the vasculature. Additionally, the stent device may include portions of the device such as side extension members which are formed of bio-erodible or bio-absorbable materials and or materials suitable for the delivery of pharmacological or therapeutic agents adapted to encourage healing during the treatment of aneurysms or reduction of plaque or restenosis during the treatment atherosclerotic lesions. Materials and coating process technology suitable for application to the present invention are described in U.S. Patent Application Publication No: 20070128723 A1 to Cottone et al., entitled, “Progenitor Endothelial Cell Capturing with a Drug Eluting Implantable Medical Device” herein incorporated by reference in its entirety.

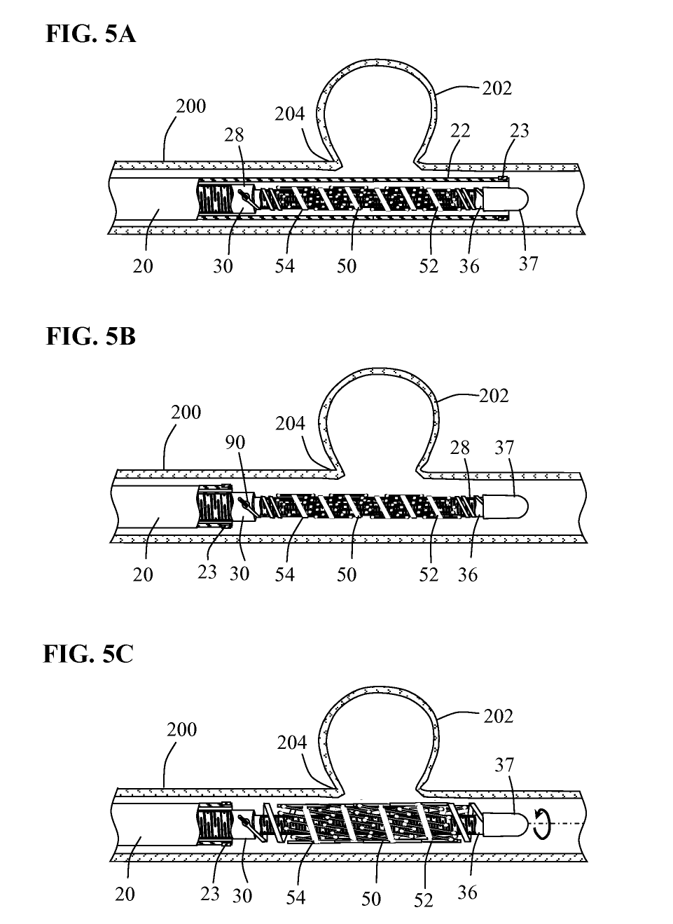

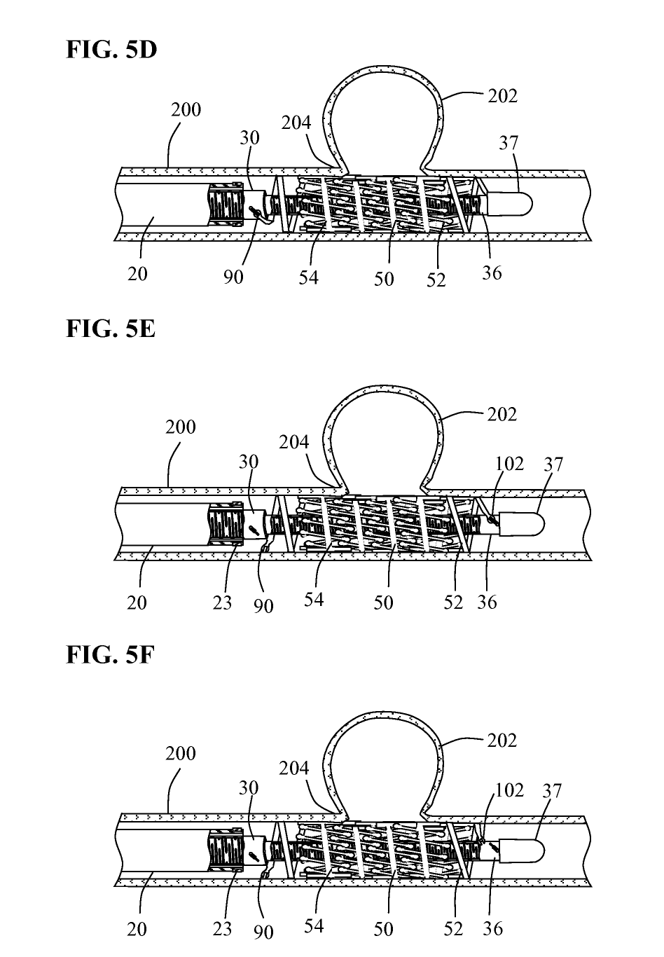

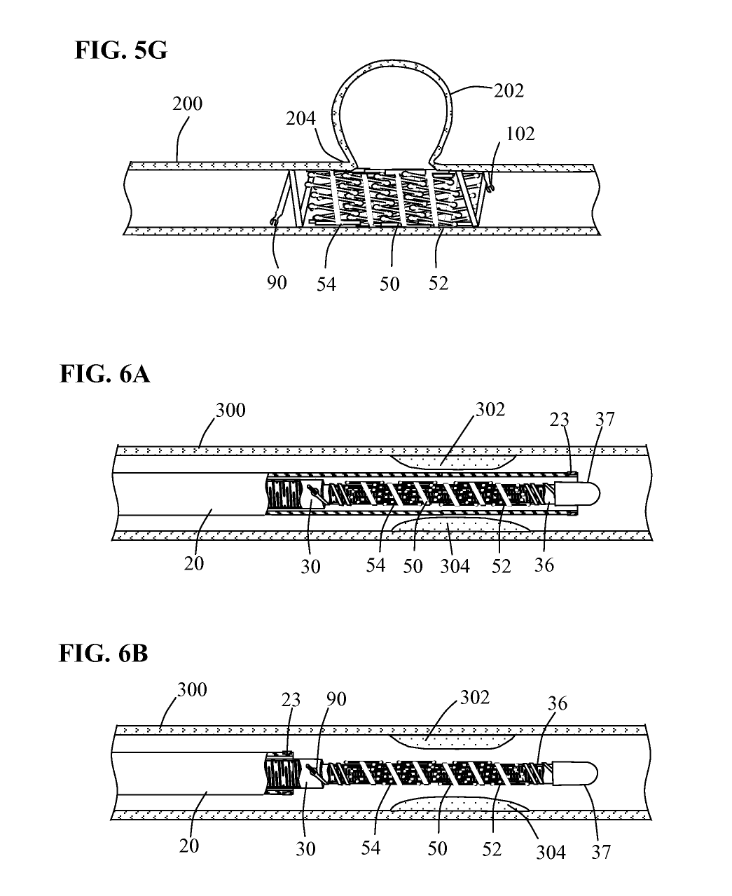

FIGS. 5A through 5F illustrate a method of deploying a stent device adjacent a vascular defect according to one embodiment of the present invention. The deployment system is positioned within a target vessel 200 having a bulging vascular defect known as an aneurysm 202. The interior of the aneurysm is coupled to the lumen of the vessel at aneurysm neck 204. The distal end of catheter 20, including stent device 50 is positioned adjacent aneurysm neck 204. Stent device 50, being in its first configuration for delivery, is wound onto and coupled to the distal end of inner member 36 and additionally coupled to outer member 30 of delivery system 28. Positioning of stent device 50 relative to aneurysm neck 204 may be aided with a radio-opaque centering marker positioned beneath the stent on inner member 36 (not shown). Catheter 20 is retracted such that catheter marker 23 is positioned proximal to proximal tab 90 of stent device 50. At the proximal end(the collective handle assembly) of deployment system 10, retainer member 45 is removed allowing axial and rotational movement of inner member 36 and outer member 30 relative to each other. As inner knob 44 is rotated relative to outer knob 42, inner member 36 rotates causing stent device 50 to unwind and expand. The expansion of stent device 50 may be controlled through the rotation and longitudinal movement of inner knob 44 relative to outer knob 42. Movement of the knobs 44 and 42 relative to each other provides the physician with the ability to control the relative proximity of side extension members positioned on adjacent winds. The expansion of stent device 50 continues until it contacts the inner wall of vessel 200. At this point in the deployment process, should the physician desire to not proceed with treating the lesion or to reposition stent device 50, knob 44 may be rotated in the opposite direction relative to knob 44 and wind stent device 50 to a reduced diameter onto inner member 36 for subsequent repositioning and redeployment or removal. Should the physician desire to release stent device 50 at the target site, power supply 60 is used to supply energy to the inner member proximal end while tip cap 37 is in a first position covering joint member 103 to cause electrolytically severable joint member 92 to sever, thereby releasing proximal tab 90 of stent device 50 from the distal end of outer member 30. Push wire 39 may then be advanced distally relative to inner member 36 causing tip cap 37 to move distally and uncover joint member 103. Energy is again supplied to the proximal end of inner member 36 from power supply 60 to cause electrolytically severable joint member 103 to sever, thereby releasing distal tab 102 of stent device 50 from delivery system 28. Delivery system 28 and catheter 20 may then be removed from the target site.

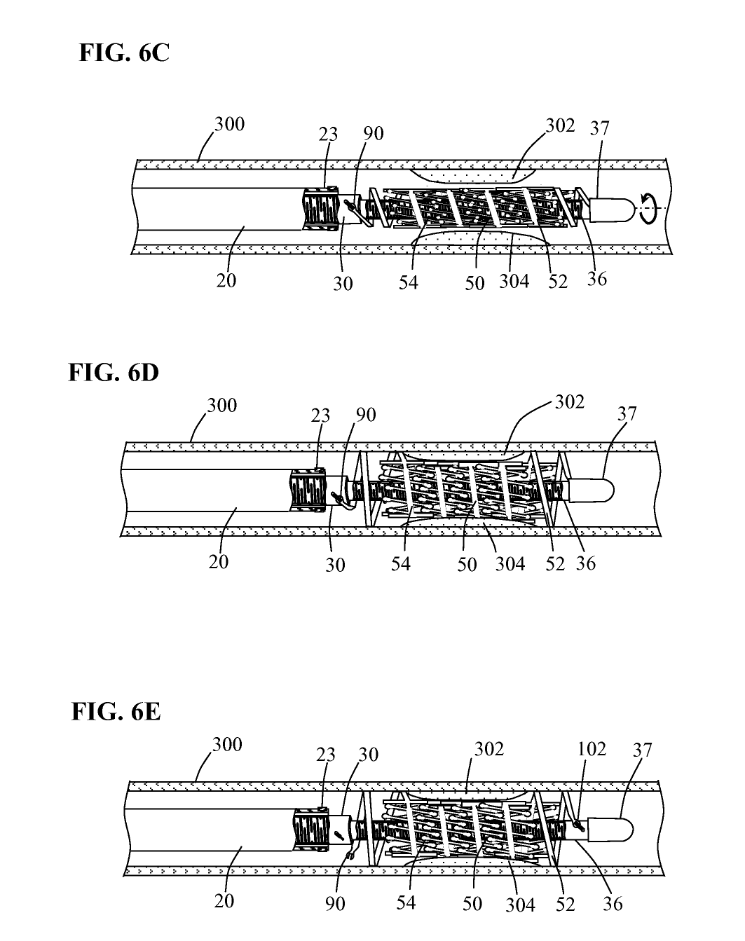

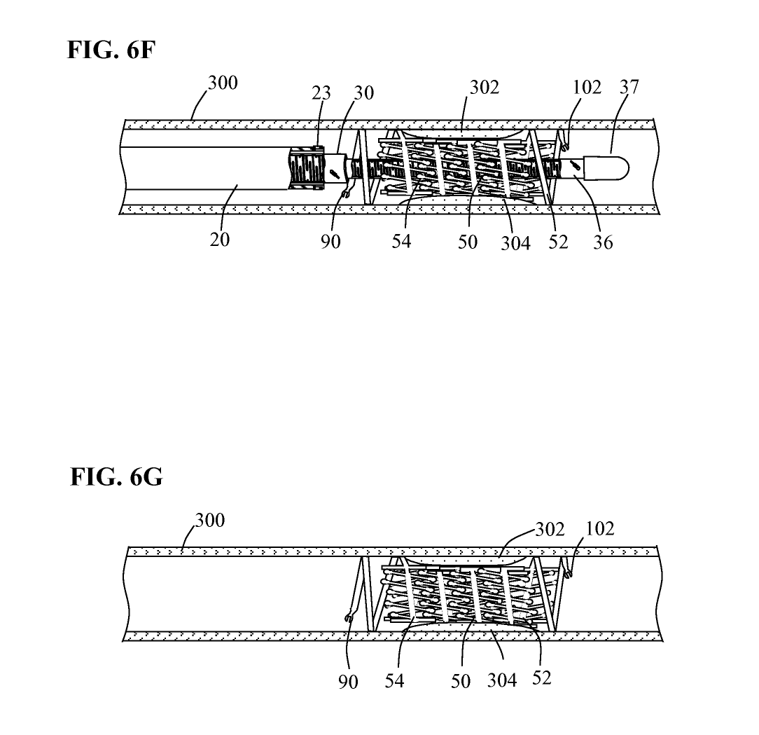

FIGS. 6A through 6F illustrate a method of deploying a stent device adjacent a vascular defect according to another embodiment of the present invention. The deployment system is positioned within a target vessel 300 having an atherosclerotic lesion comprising plaque deposits 302 and 304 creating a stenosis within the vessel restricting distal blood flow. The distal end of catheter 20, including stent device 50 is positioned adjacent plaque deposits 302 and 304. Stent device 50, being in its first configuration for delivery, is wound onto and coupled to the distal end of inner member 36 and additionally coupled to outer member 30 of delivery system 28. Positioning of stent device 50 relative to plaque deposits 302 and 304 may be aided with a radio-opaque centering marker positioned beneath the stent on inner member 36 (not shown). Catheter 20 is retracted such that catheter marker 23 is positioned proximal to proximal tab 90 of stent device 50. At the proximal end(the collective handle assembly) of deployment system 10, retainer member 45 is removed allowing axial and rotational movement of inner member 36 and outer member 30 relative to each other. As inner knob 44 is rotated relative to outer knob 42, inner member 36 rotates causing stent device 50 to unwind and being formed from a resilient material such as nitinol move from a first configuration having a reduced diameter to expand. The expansion of stent device 50 may be controlled through the rotation and longitudinal movement of inner knob 44 relative to outer knob 42. Movement of the knobs 44 and 42 relative to each other provides the physician with the ability to control the relative proximity of side extension members positioned on adjacent winds. The expansion of stent device 50 continues until it contacts the inner wall of vessel 300 distal and proximal to plaque deposits 302 and 304. At this point in the deployment process, should the physician desire to not proceed with treating the lesion or to reposition stent device 50, knob 44 may be rotated in the opposite direction relative to knob 44 and wind stent device 50 to a reduced diameter onto inner member 36 for subsequent repositioning and redeployment or removal. Should the physician desire to release stent device 50 at the target site, power supply 60 is used to supply energy to the inner member proximal end while tip cap 37 is in a first position covering joint member 103 to cause electrolytically severable joint member 92 to sever, thereby releasing proximal tab 90 of stent device 50 from the distal end of outer member 30. Push wire 39 may then be advanced distally relative to inner member 36 causing tip cap 37 to move distally and uncover joint member 103. Energy is again supplied to the proximal end of inner member 36 from power supply 60 to cause electrolytically severable joint member 103 to sever, thereby releasing distal tab 102 of stent device 50 from delivery system 28. Delivery system 28 and catheter 20 may then be removed from the target site. Although stent device 50 is in an expanded second configuration, a portion of stent device 50 may be partially constrained by plaque deposits 302 and 304. The resilient nature of stent device 50, being in an expanded configuration and slightly constrained by the lesion and vessel, creates chronic outward force which is applied to plaque deposits 302 and 304 as well as vessel 300. The chronic outward of force applied by the stent device 50 is a result of many different design features of the stent including the dimensions and geometry of the backbone or primary member, the phase transformation temperature, Af, of the nitinol used and the shape set normal unconstrained expanded diameter of the stent. When properly designed, the chronic outward force of stent device 50 allows the gradual expansion of the stent diameter in the vicinity of the plaque deposits 302 and 304 to thereby compress the plaque deposits thus reducing the restriction to blood flow in the region. Alternatively, a balloon device may be positioned within the lumen of the deployed stent device 50 and inflated to accelerate the compression of plaque deposit thereby permitting immediate revascularization.

Novel devices, systems and methods have been disclosed to perform vascular reconstruction and revascularization procedures within a mammal. Although preferred embodiments of the invention have been described, it should be understood that various modifications including the substitution of elements or components which perform substantially the same function in the same way to achieve substantially the same result may be made by those skilled in the art without departing from the scope of the claims which follow.

What is claimed is:

1. An endolumenal reconstruction device for placement in a body lumen of a mammal comprising:
an elongate primary member formed of a resilient material having a generally coiled configuration with multiple adjacent turns defining a generally helical gap between said turns;
a plurality of side extension members having first and second ends wherein only one of said ends of each side extension member is coupled to said primary member, said side extension members extending outwardly from said primary member in a generally coplanar direction such that when said primary member is in said coiled configuration at least some of said side extension members on a turn of said primary member interlock with at least some of said side extension members on an adjacent turn of said primary member.

2. A reconstruction device according to claim 1 wherein said primary member or at least one of said side extension members comprises a therapeutic compound.

3. A reconstruction device according to claim 1 wherein at least one of said side extension members is arcuate.

4. A reconstruction device according to claim 1 wherein at least one of said side extension members comprises a marker.

5. A reconstruction device according to claim 1 wherein at least one of said side extension members has an end that is tabular.

6. A reconstruction device according to claim 1 wherein said primary member has a first width at one portion of said primary member and a second width which is greater than said first width at another portion of said primary member.

7. A reconstruction device according to claim 1 wherein said primary member or at least one of said side extension members comprises a biodegradable material.

8. A reconstruction device according to claim 1 wherein said primary member has a first coiled diameter which is greater than a second coiled diameter of said primary member.

9. A reconstruction device according to claim 1 wherein said primary member has a periodic arcuate shape in addition to said generally coiled configuration.

10. A stent device for placement in a body lumen of a mammal comprising:
an elongate primary member formed of a resilient material having a generally helical configuration with multiple adjacent turns said adjacent turns defining a generally helical gap between said turns; and,
a plurality of discrete side extension members having a first end region fixedly coupled to said primary member and a second end region extending outwardly from said primary member such that when said primary member is in said helical configuration said side extension members at least partially span a portion of said helical gap to thereby form a generally tubular framework and at least some side extension members on a turn of said primary member interlock with at least some side extension members on an adjacent turn of said primary member.

11. A stent device according to claim 10 wherein said primary member or at least one of said side extension members comprises a therapeutic compound.

12. A stent device according to claim 10 wherein at least one of said side extension members is arcuate.

13. A stent device according to claim 10 wherein at least one of said side extension members comprises a marker.

14. A stent device according to claim 10 wherein at least one of said side extension members has an end that is tabular.

15. A stent device according to claim 10 wherein said primary member includes a marker.

16. A stent device according to claim 10 wherein said primary member has a first width at one portion of said primary member and a second width which is greater than said first width at another portion of said primary member.

17. A stent device according to claim 10 wherein said primary member or at least one of said side extension members comprises a biodegradable material.

18. A stent device according to claim 10 wherein the distribution of side extension members along a first portion of said primary member is greater than the distribution of said side extension members along a second portion of said primary member.

19. A stent device according to claim 10 wherein a first coiled diameter of said primary member is greater than a second coiled diameter of said primary member.

20. A stent device according to claim 10 wherein said primary member has a periodic arcuate shape in addition to said generally helical configuration.
